(19) United States
(12) Patent Application Publication
(10) Pub. No.: US 2015/0342326 A1
(43) Pub. Date: Dec. 3, 2015

(54) Personal Temperature Control and Hydration Device

(71) Applicants: Laura Drysdale, Dunedin, FL (US); Kip Shayne Drysdale, Dunedin, FL (US)

(72) Inventors: Laura Drysdale, Dunedin, FL (US); Kip Shayne Drysdale, Dunedin, FL (US)

(21) Appl. No.: 14/289,754

(22) Filed: May 29, 2014

(51) Int. Cl. A45F 3/20; A61F 7/02

(52) U.S. Cl. CPC A45F 3/20; A61F 7/02

(57) ABSTRACT

A personal temperature control and hydration device positions against a person to assist in cooling or heating the person. The device includes a cover having a closed bottom and an open top. An aperture extends through the cover. A pouch configured for holding a liquid is insertable into the cover through the open top. A tube is in fluid communication with the interior space of the pouch and insertable through the aperture when the pouch is positioned in the cover. A strap is coupled to the cover for coupling the cover to a person. Each of the cover and the pouch transmit heat wherein the cover and the pouch provide heat transfer between a liquid in the pouch and the person.

### BACKGROUND OF THE DISCLOSURE

### Field of the Disclosure

The disclosure relates to hydration devices and more particularly pertains to a new hydration device for positioning against a person to assist in cooling or heating the person.

### SUMMARY OF THE DISCLOSURE

An embodiment of the disclosure meets the needs presented above by generally comprising a cover having a closed bottom and an open top. An aperture extends through the cover. A pouch configured for holding a liquid is insertable into the cover through the open top. A tube is in fluid communication with the interior space of the pouch and insertable through the aperture when the pouch is positioned in the cover. A strap is coupled to the cover for coupling the cover to a person. Each of the cover and the pouch transmit heat wherein the cover and the pouch provide heat transfer between a liquid in the pouch and the person.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a personal temperature control and hydration device according to an embodiment of the disclosure.

FIG. 2 is a top view of an embodiment of the disclosure.

FIG. 3 is a an exploded top front side perspective view of an embodiment of the disclosure.

FIG. 4 is a front view of an embodiment of the disclosure.

FIG. 5 is a front view of an embodiment of the disclosure in use.

### DESCRIPTION OF THE PREFERRED EMBODIMENT

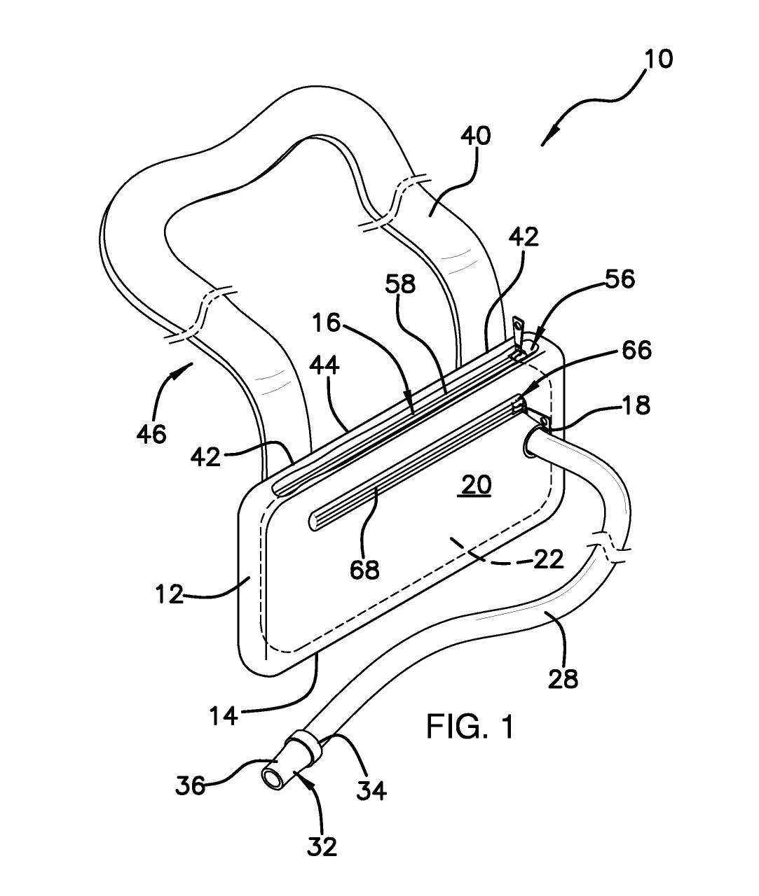

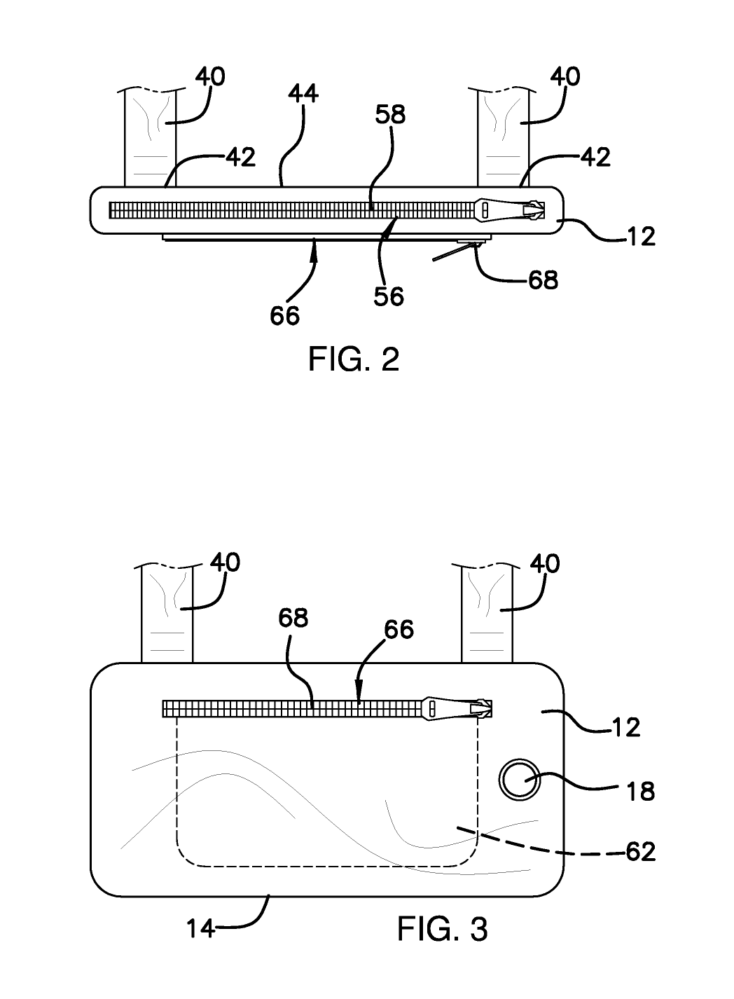

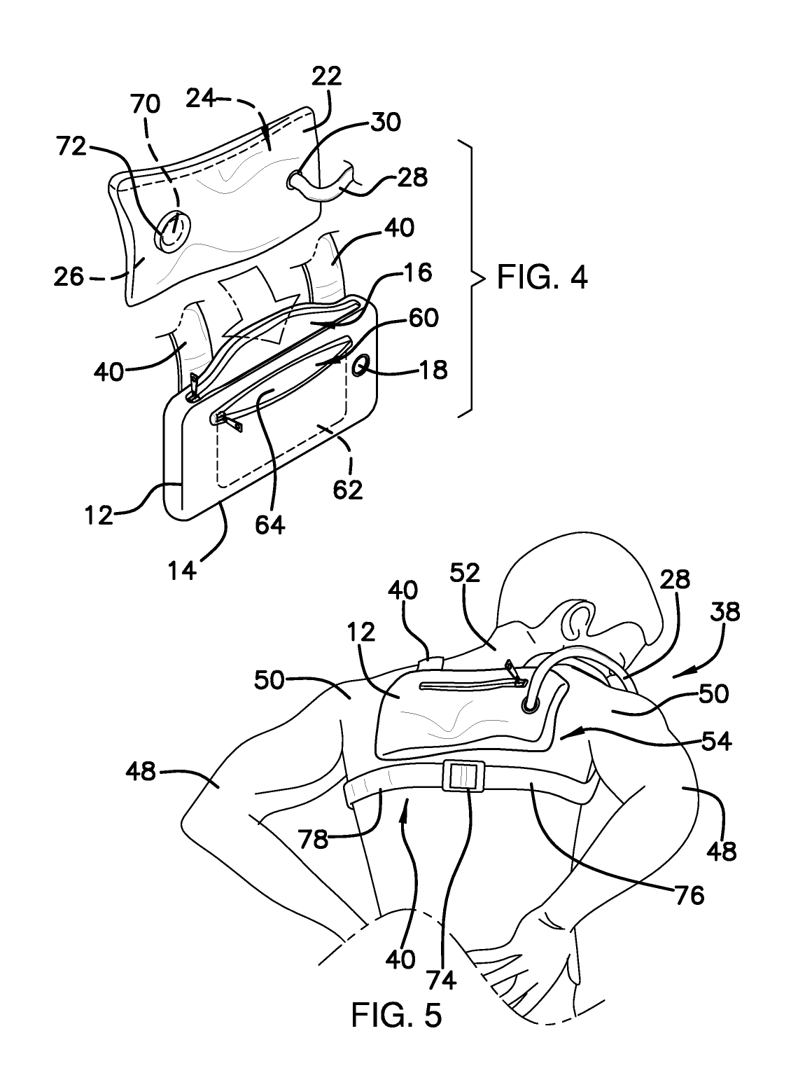

With reference now to the drawings, and in particular to FIGS. 1 through 5 thereof, a new hydration device embodying the principles and concepts of an embodiment of the disclosure and generally designated by the reference numeral 10 will be described.

As best illustrated in FIGS. 1 through 5, the personal temperature control and hydration device 10 generally comprises a cover 12 having a closed bottom 14 and an open top 16. The cover 12 is structured to comprise an aperture 18 extending through a front face 20 the cover 12. A pouch 22 has a sealable interior space 24 wherein the pouch 22 is configured for holding a liquid 26. The pouch 22 is insertable into the cover 12 through the open top 16. A tube 28 has a first end 30 coupled to the pouch 22. The first end 30 of the tube 28 is in fluid communication with the interior space 24 of the pouch 22. The tube 28 is insertable through the aperture 18 when the pouch 22 is positioned in the cover 12. A valve 32 is coupled to a distal end 34 of the tube 28 relative to the pouch 22 wherein the valve 32 is configured to prevent the liquid 26 in the pouch 22 from being distributed from the pouch 22 until a person 38 sucks on the valve 32 to draw the liquid 26 from the pouch 22. The valve 32 may have a tapered outer wall 36 to facilitate being sucked upon by the person 38.

A strap 40 is coupled to the cover 12 wherein the cover 12 is configured for coupling to the person 38. The strap 40 has opposite ends 42 coupled to an upper edge 44 of the cover 12 wherein the strap 40 defines a loop 46. The loop 46 is configured for being positioned to extend under arms 48 of the person 38, around shoulders 50 of the person 38, and behind a neck 52 of the person 38. Thusly, the pouch 22 and cover 12 are positioned on a back 54 of the person 38. A buckle 74 may be provided to selectively join a first section 76 of the strap 40 to a second section 78 of the strap. The strap 40 may have an adjustable length in a conventional manner. Each of the cover 12 and the pouch 22 transmit heat wherein the cover 12 and the pouch 22 are configured for facilitating heat transfer between the liquid 26 in the pouch 22 and the back 54 of the person 38.

A cover closure 56 is coupled to the cover 12. The cover closure 56 extends along the open top 16. The cover closure 56 closes the open top 16 wherein the pouch 22 is retained in the cover 12. The cover closure 56 may be a first slide fastener 58 such as a zipper or the like.

A slit 60 extends through the cover 12. A pocket 62 is coupled to the cover 12 aligned with the slit 60 wherein the slit 60 defines an opening 64 into the pocket 62. A pocket closure 66 is coupled to the cover 12 extending along the slit 60. The pocket closure 66 closes the opening 64 into the pocket 62. The pocket closure 66 is a second slide fastener 68 such as a zipper or the like.

A fill opening 70 extends through the pouch 22. The fill opening 70 is in fluid communication with the interior space 24 of the pouch 22 wherein the fill opening 70 is configured for facilitating putting the liquid 26 into the interior space 24. A cap 72 is removably coupled to the pouch 22. The cap 72 seals the fill opening 70 wherein the cap 72 retains the liquid 26 in the interior space 24 of the pouch 22.

In use, the person 38 fills the interior space 24 of the pouch 22 with the liquid 26. The liquid 26 may be either hot or cold as desired by the person 38. The pouch 22 is inserted into the cover 12 and the cover 12 is positioned on the person 38 as described above. The person 38 may partake of drinking the liquid 26 by sucking on the tube 28 when desired.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A personal temperature control and hydration device comprising:
a cover having a closed bottom and an open top, said cover being structured to comprise an aperture extending through said cover;
a pouch having a sealable interior space wherein said pouch is configured for holding a liquid, said pouch being insertable into said cover through said open top;
a tube having a first end coupled to said pouch, said first end of said tube being in fluid communication with said interior space of said pouch, said tube being insertable through said aperture when said pouch is positioned in said cover;
a strap coupled to said cover wherein said cover is configured for coupling to a person; and
wherein each of said cover and said pouch transmit heat wherein said cover and said pouch are configured for facilitating heat transfer between a liquid in said pouch and the back of the person.

2. The device of claim 1, further comprising said strap having opposite ends coupled to an upper edge of said cover wherein said strap defines a loop configured for being positioned to extend under arms of the person around shoulders of the person and behind a neck of the person wherein said pouch and cover are positioned on a back of the person.

3. The device of claim 1, further comprising a cover closure coupled to said cover, said cover closure extending along said open top, said cover closure closing said open top wherein said pouch is retained in said cover.

4. The device of claim 3, further comprising said closure being a slide fastener.

5. The device of claim 1, further comprising:
a slit extending through said cover; and
a pocket coupled to said cover, said pocket being aligned with said slit wherein said slit defines an opening into said pocket.

6. The device of claim 5, further comprising a pocket closure coupled to said cover, said pocket closure extending along said slit, said pocket closure closing said opening into said pocket.

7. The device of claim 1, further comprising a valve coupled to a distal end of said tube relative to said pouch wherein said valve is configured for prevent the liquid in said pouch from being distributed from said pouch until the person sucks on said valve to draw the liquid from said pouch.

8. The device of claim 1, further comprising a fill opening extending through said pouch, said fill opening being in fluid communication with said interior space of said pouch wherein said fill opening is configured for facilitating putting liquid into said interior space.

9. The device of claim 8, further comprising a cap removably coupled to said pouch, said cap sealing said fill opening wherein said cap retains liquid in said interior space of said pouch.

10. A personal temperature control and hydration device comprising:
a cover having a closed bottom and an open top, said cover being structured to comprise an aperture extending through said cover;
a pouch having a sealable interior space wherein said pouch is configured for holding a liquid, said pouch being insertable into said cover through said open top;
a tube having a first end coupled to said pouch, said first end of said tube being in fluid communication with said interior space of said pouch, said tube being insertable through said aperture when said pouch is positioned in said cover;
a strap coupled to said cover wherein said cover is configured for coupling to a person, said strap having opposite ends coupled to an upper edge of said cover wherein said strap defines a loop configured for being positioned to extend under arms of the person around shoulders of the person and behind a neck of the person wherein said pouch and cover are positioned on a back of the person;
wherein each of said cover and said pouch transmit heat wherein said cover and said pouch are configured for facilitating heat transfer between a liquid in said pouch and the back of the person;
a cover closure coupled to said cover, said cover closure extending along said open top, said cover closure closing said open top wherein said pouch is retained in said cover, said cover closure being a first slide fastener;
a slit extending through said cover;
a pocket coupled to said cover, said pocket being aligned with said slit wherein said slit defines an opening into said pocket;
a pocket closure coupled to said cover, said pocket closure extending along said slit, said pocket closure closing said opening into said pocket, said pocket closure being a second slide fastener;
a valve coupled to a distal end of said tube relative to said pouch wherein said valve is configured for prevent the liquid in said pouch from being distributed from said pouch until the person sucks on said valve to draw the liquid from said pouch;
a fill opening extending through said pouch, said fill opening being in fluid communication with said interior space of said pouch wherein said fill opening is configured for facilitating putting liquid into said interior space; and
a cap removably coupled to said pouch, said cap sealing said fill opening wherein said cap retains liquid in said interior space of said pouch.
